(19) United States
(12) Patent Application Publication
(10) Pub. No.: US 2011/0092818 A1
(43) Pub. Date: Apr. 21, 2011

(54) ULTRASONOMETER FOR BONE ASSESSMENT IN INFANTS

(75) Inventor: Armen P. Sarvazyan, Lambertville, NJ (US)

(73) Assignee: ARTANN LABORATORIES, INC., Lambertville, NJ (US)

(21) Appl. No.: 12/580,516

(22) Filed: Oct. 16, 2009

(51) Int. Cl. A61B 8/00

(52) U.S. Cl. 600/449

(57) ABSTRACT

An ultrasonometer for bone assessment in infants includes a focusing acoustic wave transducer, an acoustic wave detector, and an elongated chamber filled with an acoustically-coupling fluid. The chamber is equipped with an acoustically-transparent flexible membrane facing the extremity of the infant. Supporting means such as a gliding rod is adapted to retain at least one of the transducer or the detector inside the chamber facing the subject. Supporting means is further adapted to move the transducer or detector along the chamber to perform the bone scanning without repositioning of the probe. A focused ultrasound transducer is adapted to remotely generate an acoustic wave in the bone by acoustic radiation force.

### BACKGROUND OF THE INVENTION

The present invention relates generally to quantitative ultrasound devices for bone assessment. More particularly, the device of the invention is an ultrasonometer adapted for bone quality assessment in newborns and in infants during their first year of life.

Quantitative characterization of bone condition in pediatrics is of a great importance, particularly in neonatology, where conventional densitometry has restrictions for application in newborns and infants. The problem of assessment of newborns skeletal system became especially vital during last decades with the growing emphasis on osteopenia of prematurity—decreased bone mass and density in premature and low-birth-weight infants. Statistics shows that up to 50% of low birth weight and preterm newborns are likely to have abnormalities in bone metabolism and development. According to the National Center for Health Statistics, the incidence of pre-term birth including spontaneous and iatrogenic pre-term birth is currently over 11% in the US, the percentage of infants born of low-birth-weight is 7.6%, which means that up to 5 newborns from 100 have some form of bone pathology. Incidence of neonatal hypocalcaemia is estimated to be present in up to 30% of infants with very low birth weight (<1,500 g) and up to 89% of infants whose gestational age at birth is less than 32 weeks. Bone monitoring from neonatal period could assist in early prediction and prevention of rickets. Other pathologic conditions affecting bones in newborns and infants include: osteogenesis imperfecta, osteopetrosis, osteo-chondromatosis, inherited displasias, osteomyelitis, renal osteodystrophy, hypoparathyroidism, and others that occur with varied incidence. The main complications emanating from bone weakness are bones deformities caused by their softening, in particular, bowing of long bones and fracturing. As most of bone disorders may develop asymptomatically, they often are not noticed unless the bone is already seriously damaged. This in turn may lead to traumatic or non-traumatic fracture, stunted growth (if the growth plate is involved) and seriously worsen the patient's condition. The first crucial objective is careful and cautious handling of infants that were indicated to be at risk for these pathological conditions. The next step is enhancement of diagnostic accuracy and treatment efficiency by real-time diagnostic feedback, so that the therapy can be quickly applied and corrected if necessary as soon as positive or negative dynamics is observed.

At the present time, despite wide use of clinical radiology, quantitative assessment of bones in infants is not a routine clinical procedure because of lack of tools for screening and monitoring of infants bones by fast, convenient and safe way. The following is a brief description of available bone evaluation methods:

Plain Radiography. Plain skeletal radiography successfully demonstrates morphological changes in infants' bones that occur in different types of skeletal pathology such as shadowing of poorly mineralized areas, delays in development of bone growth point, and bone deformities like bowing of long bones. In postnatal period, plain radiograms are often taken if clinical symptoms of skeletal diseases are evident to assure the diagnosis and to undertake urgent measures. Being relatively insensitive to variations in bone density within 30-40% variation, it is not very useful for quantitative evaluation of infant bone mass or true bone density and as well for bone monitoring due to the: bulky stationary equipment, labor-consuming examinations and, the most importantly, due to the significant X-ray exposure.

Dual-Energy X-ray Absorption (DEXA). Setting a “gold standard” for osteopenia evaluation in adults, DEXA also demonstrated good sensitivity in assessing lowered bone mass in pre-term infants using spine and whole body modes. One of major drawbacks is that the child must be in a state that allows moving her/him into the scanner and, consequently, low birth weight pre-term neonates, a group at particular risk of mineral compromise, cannot be examined. To overcome this drawback, efforts were made to create a portable DEXA analyzer that can be used in the infant incubator. To make DEXA a preferred method to assess infant bone mineral content, a myriad of pitfalls should be surmounted, including considerations of bone size, growth dynamics and errors due to body fat and fat-free mass non-uniform distribution. Because of these and other reasons such as bulky equipment and high costs, DEXA still remains at the stage of clinical research for newborns despite its speed, precision and minimal radiation exposure. Advanced bone imaging technique, Quantitative Computed Tomography (QCT) does not differentiate cortical and trabecular bone components in infants while demonstrating general decreased density in preterm infants. In general, bone densitometry has not become a practical clinical instrument in neonatology at the present time.

Ultrasonography. Conventional ultrasonography was successfully tried to image skeletal abnormalities and congenital defects in newborns like displasias and dislocations of a hip. At the same time, it does not provide with quantitative characterization of bones.

Quantitative Ultrasound (QUS). Quantitative ultrasonometry devices complement radiological scanners in clinical settings with limited access to densitometers. Ultrasonic parameters of materials—ultrasound velocity and broadband attenuation, are closely related to their mechanical properties, such as elasticity moduli, and changes of structure, making the QUS technique potentially more informative as to mechanical features than X-ray techniques. Key advantages of QUS also include absence of ionizing radiation, portability, ease of use, and low cost. Ultrasonic devices for assessment of bones are divided into two groups: heel ultrasonometers, based on ultrasound through-transmission measurements of a heel bone, and axial ultrasonometers, which use surface transmission measurements in long bones such as the tibia and radius. At the present time, commercial models of bone QUS are targeted mainly at adult population, being poorly adapted to pediatric purposes with practically no application to small children and infants.

The need therefore exists for a tool for adequate diagnostics and monitoring bone health in newborns and infants.

### SUMMARY OF THE INVENTION

Accordingly, it is an object of the present invention to overcome these and other drawbacks of the prior art by providing a novel bone ultrasonometer adapted for use in newborns and small children.

It is another object of the present invention to provide a bone ultrasonometer capable of generating acoustic waves in a bone remotely using ultrasound radiation forces.

It is another object of the present invention to provide an infant bone ultrasonometer allowing scanning of the long bones of a subject without repositioning the device.

It is a further object of the present invention to provide an infant bone ultrasonometer capable of completing the entire scanning procedure in a short period of time and with minimal compression of the subject's extremity.

The infant bone ultrasonometer of this invention is in a class of devices generally adapted to perform a Quantitative Ultrasound (QUS) technique. This particular device is specifically adapted for bone quality assessment in newborn infants and infants during their 1^{st} year of life. This device is designed to assist with diagnostics and monitoring of widely spread skeletal abnormalities and diseases such as osteopenia of prematurity, different forms of osteogenesis imperfecta, and rickets. The device is intended for examination of long bones of infants, i.e. tibia, radius and ulna, and will provide information on bone growth, ossification and related pathology. Diagnostics using this device is based on analysis of axial distribution of acoustic parameters depending on elastic, geometric and structural properties of bones. Adaptation of a generally known bone ultrasonometer concept to neonatology applications requires taking into account substantial differences between the adults and infant bones: much smaller sizes, different physical properties, and fast dynamics of bone changes during the first period of life. In addition, it is essential to provide much more gentle attachment of ultrasonic transducers to the body. One of the novel features of the invention is minimization of the mechanical stress on tested site of infant's body during the measurements. It is achieved by remote generation and measurement the propagation parameters of acoustic waves through the acoustical coupling liquid filling the chamber contacting the tested site. The acoustical waves are generated remotely by a radiation force of an ultrasound beam focused on the tested bone. Description of remote generation of acoustic waves in tissue can be found in U.S. Pat Nos. 5,606,971 and 5,678,565 incorporated herein in their entirety by reference. Radiation force of a focused ultrasound wave results from the interaction of an acoustic wave with an obstacle, such as bone, placed along its path. Acoustic radiation force is produced by a change in momentum of the propagating wave and is commonly used for measurement of ultrasonic power in liquids and for calibration of therapeutic transducers.

An ultrasonometer for bone assessment of a subject utilizing remote generation of acoustic waves using acoustic radiation force according to the invention includes the following main components:

a focusing acoustic wave transducer,

an acoustic wave detector,

an elongated chamber filled with an acoustical coupling fluid. The chamber is preferably equipped on the bottom with an acoustically-transparent flexible membrane facing the extremity of the subject. Supporting means such as a gliding rod is adapted to retain at least one of the transducer or the detector inside the chamber facing the subject. Supporting means is further adapted to move the transducer or detector along the chamber, either manually or automatically to perform the bone scan.

As a result of this arrangement, once the chamber of the device is placed on the subject, an ultrasound bone scanning is performed by energizing the transducer and the detector and moving at least one of them along the bone while still inside the chamber, therefore avoiding repositioning of the device on the subject.

Other useful and distinct features of the invention include detection and analysis of different modes of ultrasonic waves propagating in bone to differentiate contributions of bone geometry and its mechanical properties and using a scanning mode of measurement, which provides axial profiles of bone acoustic parameters characterizing ossification process.

There are several important technical features that make the present invention distinctly different from and superior to existing QUS device:

remote generation of acoustic oscillations directly on the surface of the tested bone using radiation force of focused ultrasound, which minimizes the error due to contribution of soft tissues in the measured parameters;

detection of different modes of acoustical oscillation in the bone by frequency-selective analysis, which allows separating contributions of geometrical and mechanical properties of the bone into the measured parameters;

scanning mode of measurement, which enables obtaining topographical distribution of mechanical properties along the bone and visualizing spatial profiles of infant's bone ossification.

### BRIEF DESCRIPTION OF THE DRAWINGS

A more complete appreciation of the subject matter of the present invention and the various advantages thereof can be realized by reference to the following detailed description in which reference is made to the accompanying drawings in which:

FIG. 1 is a schematic illustration of the infant bone ultrasonometer according to the first embodiment of the invention,

FIG. 2 is a schematic illustration of the infant bone ultrasonometer according to the second embodiment of the invention,

FIG. 3 is a schematic illustration of the infant bone ultrasonometer according to the third embodiment of the invention,

FIG. 4 is a schematic illustration of the infant bone ultrasonometer according to the fourth embodiment of the invention,

FIG. 5 is a general illustration of the ultrasonometer of the invention in use,

FIG. 6 is a graph depicting various acoustic waves detected using the ultrasonometer of the invention, and

FIG. 7 is a schematic block diagram of the ultrasonometer of the invention.

### DETAILED DESCRIPTION OF THE PREFERRED EMBODIMENTS OF THE INVENTION

A detailed description of the present invention follows with reference to accompanying drawings in which like elements are indicated by like reference letters and numerals.

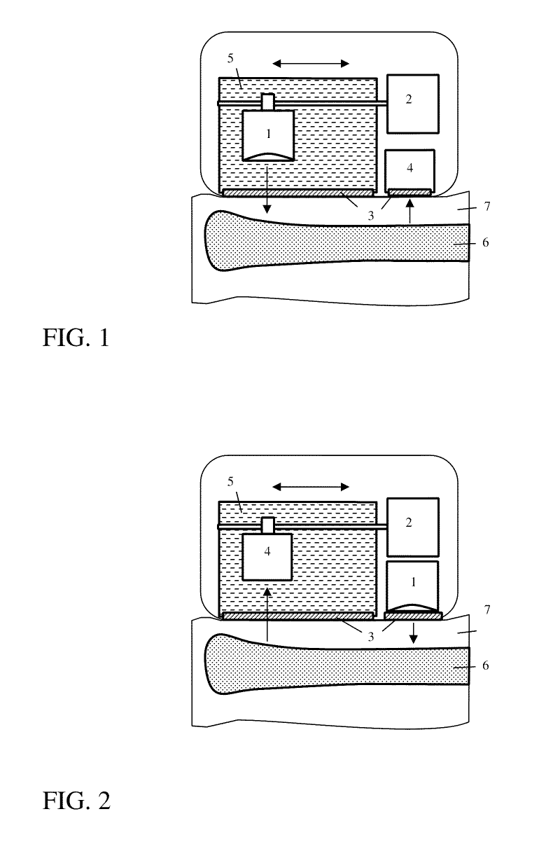

Schematic illustration of the first embodiment of the invention is found in FIG. 1. The ultrasonometer probe comprises the following main elements: focusing acoustic wave transducer 1, supporting means 2, acoustically-transparent flexible membranes 3, detector of acoustic waves 4, and an elongated chamber 5 filled with an acoustically-coupling fluid.

The acoustic wave focusing transducer 1 is preferably a simple concave piezoceramic transducer manufactured as a part of a spherical shell. Alternatively, a flat or plane piezoceramic disc with an attached acoustic lens can be used. Focusing transducer can also be made in the form of a phased array, for example an annular phased array. The individual elements of the phased array are fed from separate generators which provide phase relationships of the acoustical signals over the array aperture necessary for focusing acoustic wave at the desired distance. The focusing transducer is designed to ensure that the bone of interest will be located in its focal area when the probe is placed on the extremity of the subject.

To conduct the scan of the bone it is necessary to move either a transducer or a detector or in some cases both of them along the bone of the subject. Physical repositioning of the probe along the extremity may be detrimental to the delicate skin of the infant. To avoid this action, the present invention includes an elongated chamber 5 filled with water or another acoustically-coupling fluid and the movements of the active elements of the probe are done inside this chamber.

To ensure adequate penetration of acoustic waves into soft tissue 7 of the subject and to initiate certain useful oscillations in the bone 6, the ultrasonometer probe is equipped with a flexible acoustically transparent membrane 3 (having one or two portions depending on a particular configuration). The membrane 3 is placed on the side of the probe facing the subject. Placing the probe on the subject brings the membrane 3 in close contact with the skin and ensures a good transmission of acoustic waves generated by the focusing transducer through the soft tissues and towards the subject's bone. Preferably a polymer film with the thickness less than 0.1 mm can be used as the membrane. The material of the membrane should be biocompatible and non-toxic, such as for example that disclosed in U.S. Pat. No. 6,500,549.

Supporting means 2 are adapted to support the transducer 1 inside the chamber 5 and move it along the bone of the subject. In one embodiment, it is a sliding or threaded rod rotated manually via a crank (not shown) or automatically with an electrical motor. Rotation of the rod in one direction causes the transducer to move towards one side of the chamber, while reversing the direction of rotation causes a corresponding reversal of the movement of the transducer. The transducer itself is mounted on the supporting rod in such a way as to direct the acoustic waves towards the membrane 3 and therefore towards the subject.

As can be readily appreciated by those skilled in the art, other mechanical designs of supporting means would also make it possible to suspend the transducer 1 inside the chamber 5 while allowing it to move reciprocally back and forth therewithin.

The elongated chamber 5 is sized appropriately to allow the transducer 1 to travel along the full length of the bone. It is anticipated that the probe may come in different sizes to accommodate a range of patients and a range of extremities that can be evaluated using the ultrasonometer of the present invention. Adjustable length versions of the probe are also contemplated to be within the scope of the invention.

The detector of acoustic waves 4 may reside outside of the chamber and be equipped with its own portion of the flexible membrane 3 filled with water or other acoustical coupling fluid as shown in FIG. 1. Alternatively, it can be mounted inside the elongated chamber 5 so that a single membrane 3 is used for both the transducer 1 and the detector 4. This configuration is not shown on the drawings.

An ultrasonic Doppler vibrometer can be used as a detector of acoustic waves 4. Alternatively, conventional focusing concave piezoceramic transducers manufactured as a part of a spherical shell can be used for this purpose.

Since both the focusing transducer 1 and the detector of acoustic waves 4 are electrically activated, it is envisioned that a number of electrical cables would be routed within the probe housing to properly energize these and any other electrical devices residing in or on the probe (such as communication lights, drive motor, alarms etc.)

The second embodiment of the invention is shown in FIG. 2. The difference between this and the first embodiment is that here it is the detector 4 which is suspended inside and moved along the elongated chamber 5, while the focused transducer 1 is fixed in a stationary position within the probe of the ultrasonometer, which can be done inside (not shown) or outside the chamber 5 (shown on FIG. 2). The rest of the system is similar to the first embodiment of the invention.

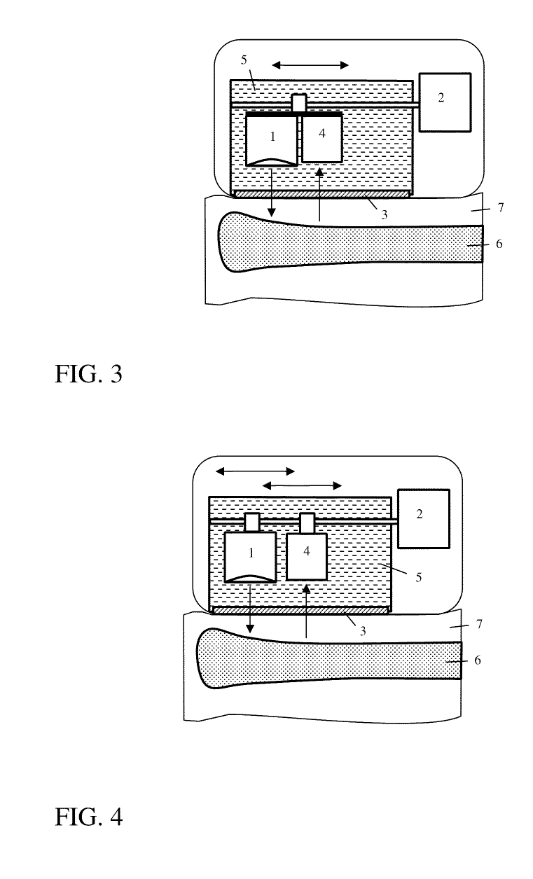

The third embodiment of the invention (FIG. 3) uses the same components as in previous embodiments but here both the focused transducer 1 and the acoustic waves detector 4 are mounted together on supporting means 2 at a fixed distance from each other. Supporting means 2 is further adapted to reciprocally move both the transducer 1 and the detector 4 together to allow for bone scanning to take place.

The fourth embodiment of the invention (FIG. 4) allows for both bone scanning and bone profiling by adapting the supporting means 2 to move the transducer 1 and the detector 4 along the same sliding rod. Thus this embodiment allows performing two types of bone examination: the one realized by the first embodiment of the invention and also the type of examination realized using the third embodiment of the invention. Using two types of bone examination in sequence may provide additional data for evaluating axial profiles of acoustical parameters of long bone.

To complete the system for infant bone ultrasonometer, additional elements are envisioned to work with the above described embodiments of the probe itself. One advantageous system comprising the probe and an electronic box further attached to a personal computer or another data storage and analysis device as shown schematically in FIG. 5.

An ultrasonic pulse transmitted along the bone travels in the form of longitudinal, surface, and various types of guided acoustic waves. These modes of acoustic waves are differentially sensitive to the mechanical (hardness), micro-structural (porosity), and geometrical or macro-structural (cortical thickness) properties of bone. To separate the contribution of the different modes of acoustic wave in the received signals and to evaluate their propagation parameters, it is necessary to analyze propagation parameters of waves at different frequencies. Different spectral components of the received broadband signal have different sensitivity to variations of cortical thickness and mineral density of long bones. At the frequencies in the range of hundreds of kHz and higher, ultrasound propagates in bone mainly as a longitudinal wave with velocity in the range of 3-4 km/s. This velocity and is defined mainly by bulk elasticity modulus of bone material.

At lower frequencies, when the acoustic wavelength becomes greater than the cortical layer thickness, acoustical energy propagates in the form of so-called guided waves which involve various complex modes of bone oscillation depending on the shape and thickness of the bone components. Guided wave velocity is greatly sensitive to changes in geometrical properties of bone, is significantly slower than the longitudinal wave velocity and its value may vary in the range from 0.8 to 2 km/s.

In use, the set of the measured parameters includes:

Velocity of the acoustic wave in the bone at the frequencies above 300 kHz, which reflects mainly mechanical properties of the bone material such as bulk modulus related to bone composition and mineralization. The velocity measured at these frequencies is close to the velocity of longitudinal wave velocity in infinite media and will be referred to as “longitudinal wave velocity”;

Velocity of acoustical wave in bone at lower frequencies, typically in the range from 70kHz to 150 kHz, which significantly depends on its geometrical characteristics of bone. In this low frequency range, the acoustical wavelength is much greater than the thickness of the cortical layer and the velocity becomes greatly dependent on the cross-sectional dimensions of the bone and its components. This velocity is referred to as the guided wave velocity;

Frequency slope of attenuation, which contains additional information on bone visco-elasticity reflecting its micro-architecture and composition;

Axial profiles of all measured acoustical parameters of bone obtained by scanning the bone from the epiphyseal cut to the middle of diaphysis. Normalized axial profiles of acoustical properties of bones contain additional diagnostic information on the bone biomechanical features. It is easier to take into account individual variations of bone parameters and make more reliable diagnostic conclusions using the spatial profile rather than just absolute values of acoustic parameters.

According to one aspect of the invention, while scanning over bone, the focusing transducer 1 periodically transmits short (preferably from 0.1 to 1 ms) pulses of ultrasound in the MHz range. The carrier frequency of the ultrasonic pulse generating vibrations in bone by acoustic radiation force is preferably in the range fro 1 to 10 MHz. Acoustical signal generated in the bone propagates through the bone and is detected by a detector 4.

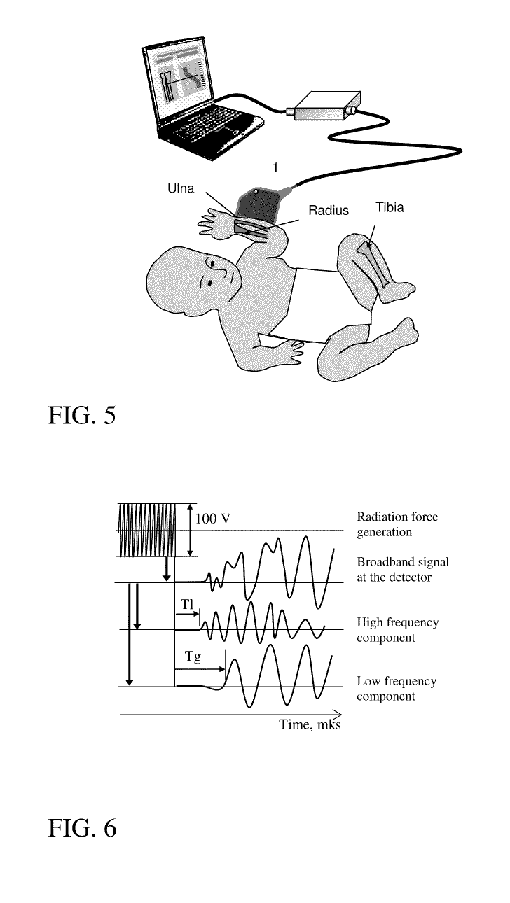

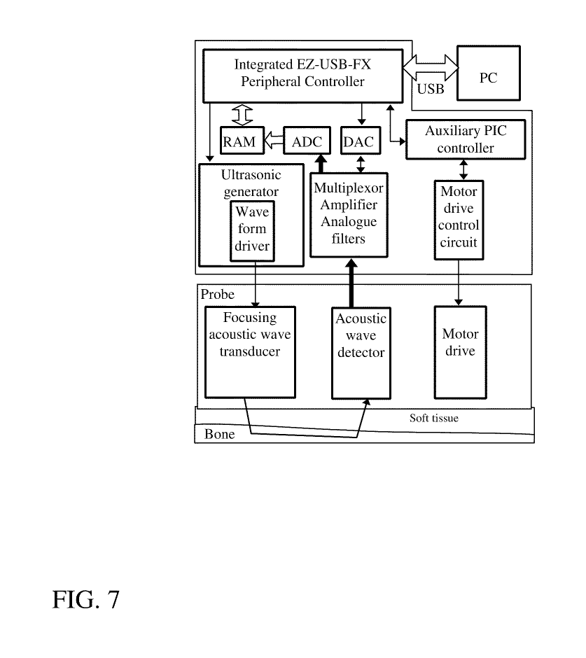

The broadband signal is received by the detector 4, amplified, digitized and processed by a series of pass band filters. The first stage of filtering removes high carrier frequency of ultrasound signal, which propagates through the surrounding tissues and the bone and interferes with the informative acoustic signal. Then two pass band filters separate spectral components in the range of 50-200 kHz (“low frequency” signal) and in the range of 300-1000 kHz (“high frequency” signal) as it is schematically shown in FIG. 6. Filtered high frequency signal serves for the measurement of the pulse time-of-flight T₁ related to the longitudinal acoustic wave propagation velocity, and, correspondingly, the low frequency signal serves for the measurement of the pulse time-of-flight T_{g} related to the guided acoustic wave propagation velocity. Frequency slope of attenuation is derived from the power spectrum by fast Fourier transform (FFT) applied to the broadband response. The scanning rate can be set in the range from 5 to 20 mm/s and the time for complete examination could be under 10 seconds. Measurements of acoustic parameters profiles along the bone will be made with the steps of about 2 mm while duration of the measurement at a single point will take several milliseconds.

In an alternative mode of operation of the device according to the invention, the radiation force generating acoustic waves in bone are produced by an amplitude modulated ultrasound pulse. As a result, the radiation force of focused ultrasound induces vibration on the bone with the frequency corresponding to the modulation frequency. The carrier frequency of the ultrasound pulse is set preferably in the range 2-10 MHz. The frequency of the amplitude modulation is selected preferably from the range 50-200 kHz for the “low frequency” acoustic wave and preferably from the range 300-600 kHz for the “high frequency” acoustic wave to be generated in the bone. Duration of the amplitude modulated ultrasonic pulse is selected preferably from the range of about 0.1 to about 1 ms. In this mode of operation, there is no need to make selective filtering of low and high frequency components of the received acoustic signal because the low and high frequency wave propagation parameters are measured not simultaneously but one after another.

Although the invention herein has been described with respect to particular embodiments, it is understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

What is claimed is:

1. An ultrasonometer for bone assessment of a subject, said ultrasonometer comprising:
a focusing acoustic wave transducer,
an acoustic wave detector,
an elongated chamber filled with an acoustically-coupling fluid, said chamber including an acoustically-transparent flexible membrane facing said subject, said chamber containing a supporting means adapted to retain at least one of said transducer or said detector within said chamber facing said subject while allowing for moving thereof along said chamber,
whereby once said chamber is placed on said subject, said bone assessment is performed by energizing said transducer and said detector and moving at least one of said transducer or said detector along the bone inside the chamber without repositioning said chamber on the subject.

2. The ultrasonometer as in claim 1, wherein said transducer is mounted on said supporting means and moved during said scanning while said detector is fixedly positioned inside said chamber.

3. The ultrasonometer as in claim 1, wherein said transducer is mounted on said supporting means and moved during said scanning while said detector is fixedly positioned outside said chamber.

4. The ultrasonometer as in claim 1, wherein said detector is mounted on said supporting means and moved during said scanning while said transducer is fixedly positioned inside said chamber.

5. The ultrasonometer as in claim 1, wherein said detector is mounted on said supporting means and moved during said scanning while said transducer is fixedly positioned outside said chamber.

6. The ultrasonometer as in claim 1, wherein both the detector and the transducer are mounted on said supporting means at a predetermined distance from each other and moved together during said scanning.

7. The ultrasonometer as in claim 1, wherein said supporting means are adapted to move said transducer and said detector independently from each other along said chamber during said scanning.

8. An ultrasonometer for bone assessment of a subject, said ultrasonometer comprising a focusing acoustic wave transducer and an acoustic wave detector, said transducer adapted for generation of a focused ultrasound wave towards the subject through an acoustically-coupling fluid, whereby during said bone assessment an acoustic wave is remotely generated in the bone of the subject by ultrasound radiation force at the site of the bone located in the focal area of said focused ultrasound wave, said acoustic wave is received by said acoustic wave detector.

9. The ultrasonometer as in claim 9, wherein said focusing transducer is a concave piezoceramic transducer.

10. The ultrasonometer as in claim 9, wherein said focusing transducer comprises a piezoceramic disk and an acoustic lens attached thereto.

11. The ultrasonometer as in claim 9, wherein said focusing transducer is a phased array transducer.

12. The ultrasonometer as in claim 9, wherein said acoustic wave detector is an ultrasonic Doppler vibrometer.
